(19) United States
(12) Patent Application Publication
(10) Pub. No.: US 2012/0039957 A1
(43) Pub. Date: Feb. 16, 2012

(54) OPTIMIZED NIACIN COMPOSITIONS IN PHARMACEUTICAL PRODUCTS

(75) Inventors: Albert W. BRZECZKO, Baltimore, MD (US); John GILKAY, Plymouth, IN (US)

(73) Assignee: ACURA PHARMACEUTICALS, INC., Palatine, IN (US)

(21) Appl. No.: 13/208,956

(22) Filed: Aug. 12, 2011

Related U.S. Application Data
(60) Provisional application No. 61/373,534, filed on Aug. 13, 2010.

(51) Int. Cl. A61K 31/455; A61K 31/485; A61P 39/00; B32B 5/16; A61K 9/14; A61P 25/04; B82Y 5/00

(52) U.S. Cl. 424/400; 514/356; 514/282; 428/402; 977/773

(57) ABSTRACT

The present invention includes compositions and methods related to forms of niacin with enhanced in-vivo absorption and bioavailability. Such enhanced delivery of niacin in the presence of food may be achieved by size reduction of crystalline niacin, use of amorphous forms of niacin, and/or pH modulation in the presence on organic acids.

### CROSS REFERENCE TO RELATED APPLICATIONS

This application claims the benefit of U.S. Provisional Application No. 61/373,534, filed Aug. 13, 2010 and is hereby incorporated by reference in its entirety.

### BACKGROUND

Niacin (commonly known as vitamin B₃) is an active ingredient in multi-vitamins, food supplements, and prescription and over-the-counter pharmaceutical products where its role is primarily as an antihyperlipidemic agent. In this capacity, niacin is understood to effect partial inhibition of free fatty acid release from adipose and increased lipoprotein lipase activity.

In addition, niacin has been extensively evaluated as an aversive agent to deter excess oral consumption of opioid analgesics in various investigational new drugs such as Acurox® with Niacin (oxycodone HCl/niacin) Tablets.

### SUMMARY OF THE INVENTION

In certain embodiments, a pharmaceutical composition includes a stable nanoparticle including niacin, wherein the nanoparticle has a particle size of about 100 nm to about 300 nm. In some embodiments, a pharmaceutical composition includes amorphous niacin.

The nanoparticle may further include surface stabilizing ingredients such as one or more of a surfactant, a powder flow aid, or a polymer. In some embodiments, the surface stabilizing ingredient includes sodium lauryl sulfate.

In some embodiments, the composition includes an organic acid. In some embodiments, the composition includes niacin and organic acid which are mixed at a particulate level, which form cocrystals, and/or at least a portion of which forms a molecular dispersion. In some embodiments, the ratio of niacin to organic acid is from about 1:5 to about 5:1, or from about 1:3 to about 3:1

In some embodiments, a composition includes an organic acid having a pKa of about 2.0 to about 6.5. The organic acid may include, for example, at least one of oxalic acid, glycine, taurine, citric acid, pyruvic acid, alanine, maleic acid, glutaric acid, or tartaric acid.

In certain embodiments, a pharmaceutical composition includes amorphous niacin, wherein the amorphous niacin comprises a solid dispersion of niacin and a polymer. In some embodiments, the polymer is a low molecular weight polymer, a water soluble polymer, and/or a polymer soluble in low pH media. In some embodiments, the polymer comprises amino methacrylate copolymer and/or poloxamer. In some embodiments, the polymer is present in an amount of up to about 1000% w/w of niacin, or about 20% w/w to about 400% w/w of niacin.

In certain embodiments, a pharmaceutical composition includes an opioid analgesic and niacin in an amount sufficient to cause flushing if greater than a prescribed amount of the analgesic of the pharmaceutical composition is ingested, wherein the niacin comprises a stable nanoparticle. In some embodiments, the composition is in unit dose form, and may be a caplet, capsule, pill, gel, soft gelatin capsule, or compressed tablet form.

In some embodiments, an analgesic is present in an amount of about 5 mg to about 200 mg on a solid weight basis. In some embodiments, the analgesic comprises hydrocodone or a therapeutically acceptable salt thereof, and/or oxycodone or a therapeutically acceptable salt thereof.

In some embodiments, a composition includes a gel forming polymer and/or a nasal tissue irritant.

In certain embodiments, a pharmaceutical composition includes a drug susceptible to abuse and niacin in an amount sufficient to cause flushing if greater than a prescribed amount of the drug of the pharmaceutical composition is ingested, wherein the niacin comprises a stable nanoparticle.

In certain embodiments, a pharmaceutical composition includes a drug susceptible to abuse and niacin in a sub-therapeutic amount, wherein the niacin comprises a stable nanoparticle.

In certain embodiments, a pharmaceutical composition includes a drug susceptible to abuse and amorphous niacin in an amount sufficient to cause flushing if greater than a prescribed amount of the drug of the pharmaceutical composition is ingested, wherein the amorphous niacin comprises a solid dispersion of niacin and a polymer. In some embodiments, a polymer includes amino methacrylate copolymer or poloxamer. In some embodiments, the polymer is present in an amount of up to about 1000% w/w of niacin, or about 20% w/w to about 400% w/w of niacin.

In certain embodiments, a pharmaceutical composition includes a drug susceptible to abuse, niacin in an amount sufficient to cause flushing if greater than a prescribed amount of the drug of the pharmaceutical composition is ingested, and an organic acid. In some embodiments, the niacin comprises a stable nanoparticle. In some embodiments, the nanoparticle includes surface quenching agents.

### BRIEF DESCRIPTION OF THE DRAWINGS

FIG. 1 shows intrinsic dissolution values of niacin samples;

FIG. 2 shows kinetic solubility of niacin samples; and

FIG. 3 shows niacin study VAS score results.

### DETAILED DESCRIPTION OF THE INVENTION

The present invention includes compositions and methods related to forms of niacin with enhanced in-vivo absorption and bioavailability. Niacin may also be known as nicotinic acid. In some embodiments, the present invention relates specifically to niacin or nicotinic acid rather than vitamin B₃.

It is believed that in the presence of food, absorption of niacin in a patient can be significantly affected. Lessened absorption may attenuate the intended abuse deterrent or therapeutic effects of niacin when niacin is taken with food. As a result, in certain embodiments this invention focuses on improved in-vivo absorption and/or bioavailability of niacin in the presence of food. In some embodiments, absorption and/or bioavailability of niacin is enhanced by improving dispersability, improving solubility and/or improving absorptive capability of niacin. In some embodiments, such enhanced delivery of niacin in the presence of food is achieved by size reduction of crystalline niacin, use of amorphous forms of niacin, and/or pH modulation in the presence on organic acids.

### A. Modified Niacin

In some embodiments, the present invention provides compositions and methods for niacin with enhanced absorption and bioavailability. Niacin absorption may be achieved when niacin is rapidly dispersed and dissolved in the upper gastrointestinal tract. In addition, it is believed that low pH of the upper gastrointestinal microenvironment may facilitate rapid and complete niacin absorption.

However, in certain instances food may slow niacin dispersibility and transiently increase upper gastrointestinal pH.

Accordingly, compositions and methods of some embodiments of the present invention relate to improving dispersability, improving solubility and/or improving absorptive capability of niacin to counteract the effect of food on the absorption and bioavailability of niacin. In some embodiments, approaches to enhancing absorption and bioavailability of niacin include (1) niacin particle size reduction, and/or (2) amorphous niacin forms, and/or (3) modifying the gastric and intestinal pH, such as for example, by coadministering niacin in the presence of organic acids to maintain a low microenvironmental pH in the gastric and intestinal mucosa, collectively “modified niacin”. In some embodiments, two or more bioenhancing approaches are used in combination.

In one embodiment, the present invention includes a dosage form including about 10 mg to about 500 mg, about 10 mg to about 600 mg, about 10 mg to about 700 mg, about 10 mg to about 800 mg, about 10 mg to about 900 mg, or about 10 mg to about 1000 mg niacin. In yet another embodiment, a dosage form of the present invention includes about 15 mg to about 150 mg niacin, or about 50 mg to about 150 mg niacin. In another embodiment, a dosage form of the present invention includes 15, 30, 45, 60, 75, 90, 105, 110, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 mg niacin. In one embodiment, the present invention includes niacin in an amount of about 1% to 25%, about 3% to 15%, or about 1%, 3%, 6%, 9%, 12%, 15%, 20%, 25%, 30%, 35%, 40%, 45%, 50%, 55%, 60%, 65%, 70%, 75%, or 80% by weight.

1. Reduced Particle Size

In some embodiments, the present invention includes reduced particle size niacin. In some embodiments, reduced particle size niacin demonstrates enhanced (a) dispersibility, (b) solubility and/or (c) absorptive capability. In some embodiments, reduced particle size niacin demonstrates enhanced absorption and/or bioavailability. In some embodiments, reduced particle size niacin demonstrates enhanced absorption and/or bioavailability independent of the presence of food.

In some embodiments, a decrease in niacin particle size results in an increase in its specific surface area and dissolution rate.

Because of the crystalline nature of niacin, in some embodiments, size reduction can be achieved by simple comminution techniques. Suitable comminution techniques include but are not limited to jet milling or wet bead milling operations. In some embodiments, dry air jet processing (micronization) is used as a suitable method for comminuting niacin. In some embodiments, dry air jet processing produces niacin with a median particle size of about 4 μm.

In some embodiments, a wet milling process is employed to produce niacin particles in the nanoparticulate range (less than about 1000 nm). In some embodiments, wet bead milling processing produces niacin nanoparticles with a median particle size of about 250 mm In some embodiments, wet bead milling processing produces niacin nanoparticles with a median particle size of about 20 nm; about 30 nm; about 40 nm; about 50 nm; about 60 nm; about 70 nm; about 80 nm; about 90 nm; about 100 nm; about 110 nm; about 120 nm; about 130 nm; about 140 nm; about 150 nm; about 160 nm; about 170 nm; about 180 nm; about 190 nm; about 200 nm; about 210 nm; about 220 nm; about 230 nm; about 240 nm; about 250 nm; about 260 nm; about 270 nm; about 280 nm; about 290 nm; about 300 nm; about 310 nm; about 320 nm; about 330 nm; about 340 nm; about 350 nm; about 360 nm; about 370 nm; about 380 nm; about 390 nm; about 400 nm; about 410 nm; about 420 nm; about 430 nm; about 440 nm; about 450 nm; about 460 nm; about 470 nm; about 480 nm; about 490 nm; about 500 nm; about 510 nm; about 520 nm; about 530 nm; about 540 nm; about 550 nm; about 560 nm; about 570 nm; about 580 nm; about 590 nm; about 600 nm; about 610 nm; about 620 nm; about 630 nm; about 640 nm; about 650 nm; about 660 nm; about 670 nm; about 680 nm; about 690 nm; about 700 nm; about 710 nm; about 720 nm; about 730 nm; about 740 nm; about 750 nm; about 760 nm; about 770 nm; about 780 nm; about 790 nm; about 800 nm; about 810 nm; about 820 nm; about 830 nm; about 840 nm; about 850 nm; about 860 nm; about 870 nm; about 880 nm; about 890 nm; about 800 nm; about 910 nm; about 920 nm; about 930 nm; about 940 nm; about 950 nm; about 960 nm; about 970 nm; about 980 nm; or about 990 nm. In some embodiments, niacin nanoparticles have a diameter of about 20 nm to about 900 nm; about 20 nm to about 875 nm; about 20 nm to about 850 nm; about 20 nm to about 825 nm; about 20 nm to about 800 nm; about 20 nm to about 775 nm; about 20 nm to about 750 nm; about 20 nm to about 725 nm; about 50 nm to about 700 nm; about 75 nm to about 675 nm; about 100 nm to about 650 nm; about 100 nm to about 625 nm; about 100 nm to about 600 nm; about 100 nm to about 575 nm; about 100 nm to about 550 nm; about 100 nm to about 525 nm; about 100 nm to about 500 nm; about 100 nm to about 475 nm; about 100 nm to about 450 nm; about 100 nm to about 425 nm; about 100 nm to about 400 nm; about 100 nm to about 375 nm; about 100 nm to about 350 nm; about 100 nm to about 325 nm; about 100 nm to about 300 nm; about 125 nm to about 300 nm; about 150 to about 300 nm; about 175 nm to about 300 nm; about 200 nm to about 300 nm; about 225 nm to about 275 nm; about 20 nm to about 300 nm; about 20 nm to about 275 nm; about 20 nm to about 250 nm; about 20 nm to about 225 nm; about 20 nm to about 200 nm; about 20 nm to about 175 nm; about 20 nm to about 150 nm; about 20 nm to about 125 nm; about 20 nm to about 100 nm; about 20 nm to about 75 nm; or about 20 nm to about 50 nm. As used herein, “particle size” is understood to mean a diameter or, for non-spherical particles, a linear measurement across the widest part of the particle. For a group of particles, the particle size is understood to represent the median particle size.

In certain embodiments, niacin is milled concomitantly with surface stabilizing ingredients. In some embodiments, surface stabilizing ingredients are desirable due to high surface energy of niacin in milling operations. In some embodiments, surface stabilizing ingredients maintain the stability of the finely divided niacin crystals. In some embodiments, surface stabilizing ingredients prevent particle agglomeration and/or crystal bridging or ripening. In some embodiments, surface stabilizing ingredients promote a more rapidly dispersing and/or rapidly dissolving reduced particle size niacin.

For dry milling processes, suitable surface stabilizing ingredients may include but are not limited to dry powers. In some embodiments, suitable stabilizing ingredients are comprised of surfactants (such as, for example, sodium lauryl sulfate) and/or powder flow aids (such as, for example, silicates including but not limited to colloidal silicon dioxide, calcium silicate, and talc).

For wet milling processes, suitable surface stabilizing ingredients may include surfactants and/or polymers. In some embodiments, sodium lauryl sulfate is an example of a suitable surfactant in wet milling processes. Other suitable surfactants may include but are not limited to polyoxyethylene sorbitan fatty acid esters such as but not limited to Polysorbate 20 and Polysorbate 80, polyoxyethylene castor oil derivatives such as but not limited to Polyoxyl 40 castor oil and Polyoxyl 60 hydrogenated castor oil, polyoxyethylene alkyl ethers such as but not limited to Cremaphor A 20 polyether and Ethylan 2560, polyoxyethylene stearates such as but not limited to Polyoxyl 100 stearate and Polyoxyl 150 distearate, polyoxylglycerides such as but not limited to lauroyl polyoxyglycerides and stearoyl polyoxyglycerides, poloxamers such as but not limited to Poloxamer 188, and sucrose fatty acid esters such as but not limited to sucrose stearate and sucrose palmitate, phospholipids and docusate sodium.

In some embodiments, suitable polymers are of low viscosity (molecular weight). In some embodiments, suitable polymers have a viscosity of about 1 mPa·s to about 25 mPa·s; about 1 mPa·s to about 20 mPa·s; about 2 mPa·s to about 18 mPa·s; about 4 mPa·s to about 16 mPa·s; about 6 mPa·s to about 14 mPa·s; or about 8 mPa·s to about 12 mPa·s. In some embodiments, suitable polymers have a viscosity of about 1 mPa·s; about 2 mPa·s; about 3 mPa·s; about 4 mPa·s; about 5 mPa·s; about 6 mPa·s; about 7 mPa·s; about 8 mPa·s; about 9 mPa·s; about 10 mPa·s; about 11 mPa·s; about 12 mPa·s; about 13 mPa·s; about 14 mPa·s; about 15 mPa·s; about 16 mPa·s; about 17 mPa·s; about 18 mPa·s; about 19 mPa·s; about 20 mPa·s; about 21 mPa·s; about 22 mPa·s; about 23 mPa·s; about 24 mPa·s; or about 25 mPa·s.

In some embodiments suitable polymers are of low molecular weight. In some embodiments, suitable polymers have a molecular weight of about 1,000 to about 50,000; about 1,000 to about 45,000; about 1,000 to about 40,000; about 1,000 to about 35,000; about 1,000 to about 30,000, about 1,000 to about 25,000; about 1,000 to about 20,000; about 1,000 to about 15,000; about 1,000 to about 10,000; about 1,000 to about 5,000; about 1,000 to about 2,500; about 2,500 to about 40,000; about 5,000 to about 40,000; about 10,000 to about 40,000; about 15,000 to about 40,000; about 20,000 to about 40,000; about 25,000 to about 40,000; about 30,000 to about 40,000; about 35,000 to about 40,000; about 2,500 to about 35,000; about 5,000 to about 35,000; about 10,000 to about 30,000; or about 15,000 to about 25,000. In some embodiments, suitable polymers have a molecular weight of about 1,000; about 1,500; about 2,000; about 2,500; about 3,000; about 4,000; about 5,000; about 6,000; about 7,000; about 8,000; about 9,000; about 10,000; about 11,000; about 12,000; about 13,000; about 14,000; about 15,000; about 16,000; about 17,000; about 18,000; about 19,000; about 20,000; about 21,000; about 22,000; about 23,000; about 24,000; about 25,000; about 26,000; about 28,000; about 30,000; about 32,000; about 34,000; about 36,000; about 38,000; or about 40,000.

In some embodiments, suitable polymers include water soluble polymers or copolymers including but not limited to copovidone, methylcellulose, carboxymethylcellulose sodium, gelatin, hydroxyethyl cellulose, hydroxyethylmethyl cellulose, hydroxypropyl cellulose, hypromellose, methylcellulose, poloxamer, polyethylene oxide, and povidone. In some embodiments, suitable polymers are soluble in low pH media such as, for example, amino methacrylate copolymer.

In some embodiments, surfactants are used during milling processes in an amount of about 1% w/w to about 100% w/w of niacin; about 1% w/w to about 90% w/w of niacin; about 1% w/w to about 80% w/w of niacin; about 1% w/w to about 70% w/w of niacin about 1% w/w to about 60% w/w of niacin; about 1% w/w to about 50% w/w of niacin; about 1% w/w to about 40% w/w of niacin; about 1% w/w to about 30% w/w of niacin; about 1% w/w to about 29% w/w of niacin; about 2% w/w to about 28% w/w of niacin; about 3% w/w to about 27% w/w of niacin; about 4% w/w to about 26% w/w of niacin; about 5% w/w to about 25% w/w of niacin; about 6% w/w to about 24% w/w of niacin; about 7% w/w to about 23% w/w of niacin; about 8% w/w to about 22% w/w of niacin; about 9% w/w to about 21% w/w of niacin; or about 10% w/w to about 20% w/w of niacin. In some embodiments, surfactants are used during milling processes in an amount of about 1% w/w of niacin; 2% w/w of niacin; 3% w/w of niacin; 4% w/w of niacin; 5% w/w of niacin; 6% w/w of niacin; 7% w/w of niacin; 8% w/w of niacin; 9% w/w of niacin; 10% w/w of niacin; 11% w/w of niacin; 12% w/w of niacin; 13% w/w of niacin; 14% w/w of niacin; 15% w/w of niacin; 16% w/w of niacin; 17% w/w of niacin; 18% w/w of niacin; 19% w/w of niacin; 20% w/w of niacin; 21% w/w of niacin; 22% w/w of niacin; 23% w/w of niacin; 24% w/w of niacin; 25% w/w of niacin; 25% w/w of niacin; 26% w/w of niacin; 27% w/w of niacin; 28% w/w of niacin; 29% w/w of niacin; 30% w/w of niacin; 31% w/w of niacin; 32% w/w of niacin; 33% w/w of niacin; 34% w/w of niacin; 35% w/w of niacin; 36% w/w of niacin; 37% w/w of niacin; 38% w/w of niacin; 39% w/w of niacin; 40% w/w of niacin; 41% w/w of niacin; 42% w/w of niacin; 43% w/w of niacin; 44% w/w of niacin; 45% w/w of niacin; 46% w/w of niacin; 47% w/w of niacin; 48% w/w of niacin; 49% w/w of niacin; 50% w/w of niacin; 51% w/w of niacin; 52% w/w of niacin; 53% w/w of niacin; 54% w/w of niacin; 55% w/w of niacin; 56% w/w of niacin; 57% w/w of niacin; 58% w/w of niacin; 59% w/w of niacin; or 60% w/w of niacin.

In some embodiments, polymers are used during milling processes in an amount of up to about 800% w/w of niacin; up to about 775% w/w of niacin; up to about 750% w/w of niacin; up to about 725% w/w of niacin; up to about 700% w/w of niacin; up to about 675% w/w of niacin; up to about 650% w/w of niacin; up to about 625% w/w of niacin; up to about 600% w/w of niacin; up to about 575% w/w of niacin; up to about 550% w/w of niacin; up to about 525% w/w of niacin; up to about 500% w/w of niacin; up to about 475% w/w of niacin; up to about 450% w/w of niacin; up to about 425% w/w of niacin; up to about 400% w/w of niacin; up to about 375% w/w of niacin; up to about 350% w/w of niacin; up to about 325% w/w of niacin; up to about 300% w/w of niacin; up to about 275% w/w of niacin; up to about 250% w/w of niacin; up to about 200% w/w of niacin; up to about 175% w/w of niacin; up to about 150% w/w of niacin; up to about 125% w/w of niacin; up to about 100% w/w of niacin; up to about 75% w/w of niacin; up to about 50% w/w of niacin; or up to about 25% w/w of niacin. In some embodiments, polymers are used during milling processes in an amount of about 25% w/w to about 600% w/w of niacin; about 25% w/w to about 575% w/w of niacin; about 25% w/w to about 550% w/w of niacin; about 25% w/w to about 525% w/w of niacin; about 25% w/w to about 500% w/w of niacin; about 25% w/w to about 475% w/w of niacin; about 25% w/w to about 450% w/w of niacin; about 25% w/w to about 425% w/w of niacin; about 25% w/w to about 400% w/w of niacin; about 25% w/w to about 375% w/w of niacin; about 25% w/w to about 350% w/w of niacin; about 25% w/w to about 325% w/w of niacin; about 25% w/w to about 300% w/w of niacin; about 25% w/w to about 275% w/w of niacin; about 25% w/w to about 250% w/w of niacin; about 25% w/w to about 225% w/w of niacin; about 25% w/w to about 200% w/w of niacin; about 25% w/w to about 175% w/w of niacin; about 25% w/w to about 150% w/w of niacin; about 25% w/w to about 125% w/w of niacin; about 25% w/w to about 100% w/w of niacin; about 25% w/w to about 75% w/w of niacin; or about 25% w/w to about 50% w/w of niacin. In some embodiments, polymers are used during milling processes in an amount of about 25% w/w of niacin; about 50% w/w of niacin; about 75% w/w of niacin; about 100% w/w of niacin; about 125% w/w of niacin; about 150% w/w of niacin; about 175% w/w of niacin; about 200% w/w of niacin; about 225% w/w of niacin; about 250% w/w of niacin; about 300% w/w of niacin; about 325% w/w of niacin; about 375% w/w of niacin; about 400% w/w of niacin; about 425% w/w of niacin; about 475% w/w of niacin; about 500% w/w of niacin; about 525% w/w of niacin; about 550% w/w of niacin; about 575% w/w of niacin; about 600% w/w of niacin; about 625% w/w of niacin; about 650% w/w of niacin; about 675% w/w of niacin; about 700% w/w of niacin; about 725% w/w of niacin; about 750% w/w of niacin; about 775% w/w of niacin; or about 800% w/w of niacin.

In some embodiments, liquid used in a wet milling process to produce nanoparticles is a dispersant with very low solubility for niacin. In certain embodiments, suitable liquid dispersants include but are not limited to the class of non-polar organic liquids such as, for example, hexanes, heptanes, cyclohexane, benzene, and others. In some embodiments, the dispersant used is a hexane.

Other size-reduction or “top-down” techniques may also be used to produce niacin nanoparticles. In some embodiments, suitable techniques include jet stream disintegration piston gap homogenization and supercritical fluids. In some embodiments, precipitation or “bottom up” techniques are employed to produce niacin nanoparticles. Suitable precipitation techniques include but are not limited to jet stream microchannel reactors.

2. Amorphous Forms of Niacin

In some embodiments, the present invention includes amorphous forms of niacin. In some embodiments, amorphous forms of niacin demonstrate enhanced dispersability, solubility and/or absorptive capability. In some embodiments, amorphous forms of niacin demonstrate enhanced absorption and/or bioavailability. In some embodiments, amorphous forms of niacin demonstrate enhanced absorption and/or bioavailability independent of the presence of food.

Niacin in an amorphous state may be in a high energy, random order and interaction with the dissolving media may be maximized. However, it is understood that niacin may prefer to revert to a low energy ordered crystalline state where dispersibility and solubility may be limited in the dissolving media.

It is understood that niacin is likely to only exist in its crystalline form because transient amorphous forms of niacin may revert to crystal morphs once crystal nuclei are formed in the solid. Thus, obtaining stable amorphous forms of niacin is generally difficult.

In some embodiments, amorphous form of niacin is produced as a solid dispersion of niacin and a polymer. In some embodiments, suitable polymers are low viscosity (molecular weight). In some embodiments, suitable polymers have a viscosity of about 1 mPa·s to about 25 mPa·s; about 1 mPa·s to about 20 mPa·s; about 2 mPa·s to about 18 mPa·s; about 4 mPa·s to about 16 mPa·s; about 6 mPa·s to about 14 mPa·s; or about 8 mPa·s to about 12 mPa·s. In some embodiments, suitable polymers have a viscosity of about 1 mPa·s; about 2 mPa·s; about 3 mPa·s; about 4 mPa·s; about 5 mPa·s; about 6 mPa·s; about 7 mPa·s; about 8 mPa·s; about 9 mPa·s; about 10 mPa·s; about 11 mPa·s; about 12 mPa·s; about 13 mPa·s; about 14 mPa·s; about 15 mPa·s; about 16 mPa·s; about 17 mPa·s; about 18 mPa·s; about 19 mPa·s; about 20 mPa·s; about 21 mPa·s; about 22 mPa·s; about 23 mPa·s; about 24 mPa·s; or about 25 mPa·s.

In some embodiments suitable polymers are of low molecular weight. In some embodiments, suitable polymers have a molecular weight of about 1,000 to about 50,000; about 1,000 to about 45,000; about 1,000 to about 40,000; about 1,000 to about 35,000; about 1,000 to about 30,000, about 1,000 to about 25,000; about 1,000 to about 20,000; about 1,000 to about 15,000; about 1,000 to about 10,000; about 1,000 to about 5,000; about 1,000 to about 2,500; about 2,500 to about 40,000; about 5,000 to about 40,000; about 10,000 to about 40,000; about 15,000 to about 40,000; about 20,000 to about 40,000; about 25,000 to about 40,000; about 30,000 to about 40,000; about 35,000 to about 40,000; about 2,500 to about 35,000; about 5,000 to about 35,000; about 10,000 to about 30,000; or about 15,000 to about 25,000. In some embodiments, suitable polymers have a molecular weight of about 1,000; about 1,500; about 2,000; about 2,500; about 3,000; about 4,000; about 5,000; about 6,000; about 7,000; about 8,000; about 9,000; about 10,000; about 11,000; about 12,000; about 13,000; about 14,000; about 15,000; about 16,000; about 17,000; about 18,000; about 19,000; about 20,000; about 21,000; about 22,000; about 23,000; about 24,000; about 25,000; about 26,000; about 28,000; about 30,000; about 32,000; about 34,000; about 36,000; about 38,000; or about 40,000.

In some embodiments, suitable polymers include water soluble polymers including but not limited to copovidone, methylcellulose, carboxymethylcellulose sodium, gelatin, hydroxyethyl cellulose, hydroxyethylmethyl cellulose, hydroxypropyl cellulose, hypromellose, methylcellulose, poloxamer polyethylene oxide, and povidone. In some embodiments, suitable polymers include polymers which are soluble in low pH media such as, for example, amino methacrylate copolymer.

Polymers are used at levels up to 1500% w/w of niacin and more preferably at 20-400% w/w. In some embodiments, polymers are used in a solid dispersion with niacin in an amount of up to about 1500% w/w of niacin; up to about 1450% w/w of niacin; up to about 1400% w/w of niacin; up to about 1350% w/w of niacin; up to about 1300% w/w of niacin; up to about 1250% w/w of niacin; up to about 1200% w/w of niacin; up to about 1150% w/w of niacin; up to about 1100% w/w of niacin; up to about 1050% w/w of niacin; up to about 1000% w/w of niacin; up to about 950% w/w of niacin; up to about 900% w/w of niacin; up to about 850% w/w of niacin; up to about 800% w/w of niacin; up to about 750% w/w of niacin; up to about 700% w/w of niacin; up to about 650% w/w of niacin; up to about 600% w/w of niacin; up to about 550% w/w of niacin; up to about 500% w/w of niacin; up to about 475% w/w of niacin; up to about 450% w/w of niacin; up to about 425% w/w of niacin; up to about 400% w/w of niacin; up to about 375% w/w of niacin; up to about 350% w/w of niacin; up to about 325% w/w of niacin; up to about 300% w/w of niacin; up to about 275% w/w of niacin; up to about 250% w/w of niacin; up to about 200% w/w of niacin; up to about 175% w/w of niacin; up to about 150% w/w of niacin; up to about 125% w/w of niacin; up to about 100% w/w of niacin; up to about 75% w/w of niacin; up to about 50% w/w of niacin; up to about 25% w/w of niacin; or up to about 20% w/w of niacin. In some embodiments, polymers are used in a solid dispersion with niacin in an amount of about 20% w/w to about 1500% w/w of niacin; about 20% w/w to about 1500% w/w of niacin; about 20% w/w to about 1450% w/w of niacin; about 20% w/w to about 1400% w/w of niacin; about 20% w/w to about 1350% w/w of niacin; about 20% w/w to about 1300% w/w of niacin; about 20% w/w to about 1250% w/w of niacin; about 20% w/w to about 1200% w/w of niacin; about 20% w/w to about 1150% w/w of niacin; about 20% w/w to about 1100% w/w of niacin; about 20% w/w to about 1050% w/w of niacin; about 20% w/w to about 1000% w/w of niacin; about 20% w/w to about 950% w/w of niacin; about 20% w/w to about 900% w/w of niacin; about 20% w/w to about 850% w/w of niacin; about 20% w/w to about 800% w/w of niacin; about 20% w/w to about 750% w/w of niacin; about 20% w/w to about 700% w/w of niacin; about 20% w/w to about 650% w/w of niacin; about 20% w/w to about 600% w/w of niacin; about 20% w/w to about 550% w/w of niacin; about 20% w/w to about 500% w/w of niacin; about 20% w/w to about 475% w/w of niacin; about 20% w/w to about 450% w/w of niacin; about 20% w/w to about 425% w/w of niacin; about 20% w/w to about 400% w/w of niacin; about 20% w/w to about 375% w/w of niacin; about 20% w/w to about 350% w/w of niacin; about 20% w/w to about 325% w/w of niacin; about 20% w/w to about 300% w/w of niacin; about 20% w/w to about 275% w/w of niacin; about 20% w/w to about 250% w/w of niacin; about 20% w/w to about 200% w/w of niacin; about 20% w/w to about 175% w/w of niacin; about 20% w/w to about 150% w/w of niacin; about 20% w/w to about 125% w/w of niacin; about 20% w/w to about 100% w/w of niacin; about 20% w/w to about 75% w/w of niacin; about 20% w/w to about 50% w/w of niacin; or about 20% w/w to about 25% w/w of niacin. In some embodiments, polymers are used in solid dispersions with niacin in an amount of about 25% w/w of niacin; about 50% w/w of niacin; about 75% w/w of niacin; about 100% w/w of niacin; about 125% w/w of niacin; about 150% w/w of niacin; about 175% w/w of niacin; about 200% w/w of niacin; about 225% w/w of niacin; about 250% w/w of niacin; about 300% w/w of niacin; about 325% w/w of niacin; about 375% w/w of niacin; about 400% w/w of niacin; about 425% w/w of niacin; about 475% w/w of niacin; about 500% w/w of niacin; about 525% w/w of niacin; about 550% w/w of niacin; about 575% w/w of niacin; about 600% w/w of niacin; about 650% w/w of niacin; about 700% w/w of niacin; about 750% w/w of niacin; about 800% w/w of niacin; about 850% w/w of niacin; about 900% w/w of niacin; about 950% w/w of niacin; about 1000% w/w of niacin; about 1050% w/w of niacin; about 1100% w/w of niacin; about 1150% w/w of niacin; about 1200% w/w of niacin; about 1250% w/w of niacin; about 1300% w/w of niacin; about 1350% w/w of niacin; about 1400% w/w of niacin; about 1450% w/w of niacin; or about 1500% w/w of niacin.

In some embodiments, surfactant is included as an adjuvant (i.e., to improve wettability and/or dispersibility). Sodium lauryl sulfate is an example of a suitable surfactant. Other suitable surfactants may include but are not limited to polyoxyethylene sorbitan fatty acid esters such as but not limited to Polysorbate 20 and Polysorbate 80, polyoxyethylene castor oil derivatives such as but not limited to Polyoxyl 40 castor oil and Polyoxyl 60 hydrogenated castor oil, polyoxyethylene alkyl ethers such as but not limited to Cremaphor A 20 polyether and Ethylan 2560, polyoxyethylene stearates such as but not limited to Polyoxyl 100 stearate and Polyoxyl 150 distearate, polyoxylglycerides such as but not limited to lauroyl polyoxyglycerides and stearoyl polyoxyglycerides, poloxamers such as but not limited to Poloxamer 188, sucrose fatty acid esters such as but not limited to sucrose stearate and sucrose palmitate, phospholipids and docusate sodium.

In certain embodiments, a stable amorphous niacin/polymer solid dispersion is formed by dissolving crystalline niacin and a polymer in a solvent or solvent system. In some embodiments, suitable solvents or solvent systems include but are not limited to dichloromethane and methanol. In some embodiments, the niacin/polymer solution is then rapidly dried. Any suitable drying technique(s) may be used in order to produce the amorphous niacin/polymer solid dispersion.

In some embodiments, spray drying is a suitable drying technique. While not wishing to be bound by theory, it is believed that when the niacin/polymer solution is sprayed into a stream of hot drying gas in a spray dryer, solution droplets are formed and the solvent or solvent system is flash evaporated, leading to an increase in polymer viscosity in the partially dried droplet. The increased polymer viscosity during drying may slow the molecular mobility of niacin and inhibit niacin molecular alignment to readily form crystals. As drying is completed, niacin may be randomly ordered and locked in the polymer matrix as an amorphous glass (at a temperature below the glass transition temperature (Tg)). The solvent or solvent system should have a high vapor pressure (low boiling point) to facilitate rapid evaporation and complete drying. In some embodiments, the solvent or solvent system comprises dichloromethane, and may have a boiling point of 40° C. with a vapor pressure of 97 KP at 20° C.

3. Modified pH

In some embodiments, the present invention includes compositions and methods of maintaining a low pH in the gastrointestinal environment or microenvironment. In some embodiments, maintenance of a low pH provides enhanced dispersability, solubility and/or absorptive capability of niacin. In some embodiments, maintenance of a low pH provides enhanced absorption and/or bioavailability of niacin. In some embodiments, maintenance of a low pH provides enhanced absorption and/or bioavailability of niacin independent of the presence of food.

It is understood that the median fasted stomach pH is about 1.7. However, when a meal is administered, the stomach pH may rapidly rise to a median peak pH value of about 6.7, and then may decline gradually back to the fasted state value in less than two hours.

It has been found that this pH effect on niacin is meaningful since the pKa for niacin is 4.85. Thus at low stomach pH, as in the fasted state, (i.e. pH=2) most of the niacin may be in the free acid form or non-ionized state and at high stomach pH as in the fed state (i.e. pH=6.5) most of the niacin will be in the charged or ionized form.

It is believed that non-ionized drugs are better absorbed than ionized form, particularly when the charge to mass ratio is “high” (niacin MW 123 g/mol). For example, it has been found that niacin can be better absorbed into the bloodstream from the gastric environment when the niacin is present in the undissociated or non-ionized form. This greater absorption of niacin notably occurs when niacin is taken on an empty stomach, i.e., when the baseline gastric pH is low (pH ˜2). However, when the same niacin dose is taken with food, it has been observed that niacin absorption into the bloodstream is notably reduced. Lower absorption of niacin may be associated with a rapid rise in stomach pH due to a buffering action from the presence of food. Furthermore it has been reported that intestinal uptake of nicotinic acid is enhanced approximately five fold when intestinal pH is reduced from pH 8 to pH 5 and thus a lower pH would favor niacin absorption in the intestine as well. While not wishing to be bound by theory, it is believed that the rise in gastrointestinal pH results in a niacin form with a resulting decrease in permeability and niacin absorption. Therefore, in some embodiments, methods and compositions of the present invention maintain a low microenvironmental pH in the gastrointestinal mucosal tissue to facilitate the permeability and/or absorption of niacin in a high pH environment such as that caused by food, achlorhydria, pancreatic secretions or other conditions.

In some embodiments, a composition of the present invention includes niacin and an organic acid. Many organic acids may be suitable for this invention, including but not limited to organic acids with a low pKa. In some embodiments, suitable organic acids may have a pKa of about 1.0 to about 7.5; about 1.5 to about 7.0; about 2.0 to about 6.5; about 2.5 to about 6.0; about 3.0 to about 5.5; about 3.5 to about 5.0; or about 4.0 to about 4.5. In some embodiments, suitable organic acids may have a pKa of about 1.0; about 1.5; about 2.0; about 2.5; about 3.0; about 3.5; about 4.0; about 4.5; about 5.0; about 5.5; about 6.0; about 6.5; about 7.0; or about 7.5.

Organic acids with a low pKa may act to buffer a low microenviromental pH in a potentially higher pH environment. Suitable organics acids may include but are not limited to oxalic acid, glycine, taurine, citric acid, pyruvic acid, alanine, maleic acid, malic acid, glutaric acid, and tartaric acid. In some embodiments, polyvalent organic acids are particularly suitable, as the secondary and/or tertiary protons may act to buffer at slightly higher pH values while maintaining niacin in a favored absorption form. It is noted that niacin is 50% non-ionized at pH 4.8. In certain embodiments, suitable organic acids include citric acid, which is triprotic (pKa's at 3.15, 4.77 and 5.19).

In some embodiments, a combination of organic acids is used to provide complimenting dissociation constants (pKa's) and varying solubility. In some embodiments, the complimenting dissociation constants and varying solubility acts to match the dissolution of niacin and maintain a low pH in the microenvironment in order to promote niacin absorption. In some embodiments, suitable ratios of niacin to organic acid are from about 1:7 to about 7:1; about 1:6 to about 6:1; about 1:5 to about 5:1; about 1:4 to about 4:1; about 1:3 to about 3:1; about 1:2 to about 2:1. In some embodiments, suitable ratios of niacin to organic acid are about 1:7; about 1:6; about 1:5; about 1:4; about 1:3; about 1:2; about 1:1; about 2:1; about 3:1; about 4:1; about 5:1; about 6:1; or about 7:1.

In certain embodiments, niacin and organic acid(s) are coprocessed such that they are in close proximity during the dissolution and absorption of niacin. In some embodiments, niacin and organic acid(s) are mixed at a particulate level. Methods for coprocessing may include dry processing and/or wet processing. Suitable dry processing methods may include but are not limited to roller compaction, slugging or melt extrusion. Suitable wet processing may include but are not limited to high shear granulation, fluid bed granulation, spray drying or supercritical fluid technology.

In some embodiments, at least a portion of niacin and organic acid(s) form a molecular dispersion.

In some embodiments, cocrystals of niacin and organic acid(s) are formed. In some embodiments, cocrystals of niacin and organic acid(s) are formed where niacin and organic acid(s) have a high degree of mix.

In certain embodiments, polymers are used to improve and sustain the low pH microenvironment of the dissolving niacin/organic acid mix. According to some embodiments, polymers are well mixed with niacin and organics acid(s) to form a matrix. In some embodiments, polymers are used to coat the niacin/organic acid(s) particles and/or granules. In some embodiments, suitable polymers include water soluble polymers such as, copovidone, methylcellulose, carbomer, carboxymethylcellulose sodium, ceratonia, gelatin, guar gum, hydroxyethyl cellulose, hydroxyethylmethyl cellulose, hydroxypropyl cellulose, hypromellose, methylcellulose, poloxamer, polyethylene oxide, povidone, sodium hyaluronate, and xanthan gum. In some embodiments, suitable polymers include polymers soluble in low pH media such as, for example, amino methacrylate copolymer.

In certain embodiments, polymers are included at levels of about 5% w/w to about 500% w/w of the niacin/organic acid mix; about 5% w/w to about 475% w/w of the niacin/organic acid mix; about 5% w/w to about 450% w/w of the niacin/organic acid mix; about 5% w/w to about 425% w/w of the niacin/organic acid mix; about 5% w/w to about 400% w/w of the niacin/organic acid mix; about 5% w/w to about 375% w/w of the niacin/organic acid mix; about 5% w/w to about 350% w/w of the niacin/organic acid mix; about 5% w/w to about 325% w/w of the niacin/organic acid mix; about 5% w/w to about 300% w/w of the niacin/organic acid mix; about 5% w/w to about 275% w/w of the niacin/organic acid mix; about 5% w/w to about 250% w/w of the niacin/organic acid mix; about 5% w/w to about 225% w/w of the niacin/organic acid mix; about 5% w/w to about 200% w/w of the niacin/organic acid mix; about 25% w/w to about 175% w/w of the niacin/organic acid mix; about 50% w/w to about 150% w/w of the niacin/organic acid mix; about 75% w/w to about 125% w/w of the niacin/organic acid mix; about 5% w/w to about 100% w/w of the niacin/organic acid mix; about 5% w/w to about 75% w/w of the niacin/organic acid mix; about 5% w/w to about 50% w/w of the niacin/organic acid mix; or about 5% w/w to about 25% w/w of the niacin/organic acid mix. In certain embodiments, polymers are included at levels of about 5% w/w of the niacin/organic acid mix; about 10% w/w of the niacin/organic acid mix; about 15% w/w of the niacin/organic acid mix; about 20% w/w of the niacin/organic acid mix; about 25% w/w of the niacin/organic acid mix; about 30% w/w of the niacin/organic acid mix; about 40% w/w of the niacin/organic acid mix; about 50% w/w of the niacin/organic acid mix; about 60% w/w of the niacin/organic acid mix; about 70% w/w of the niacin/organic acid mix; about 80% w/w of the niacin/organic acid mix; about 90% w/w of the niacin/organic acid mix; about 100% w/w of the niacin/organic acid mix; about 125% w/w of the niacin/organic acid mix; about 150% w/w of the niacin/organic acid mix; about 175% w/w of the niacin/organic acid mix; about 200% w/w of the niacin/organic acid mix; about 225% w/w of the niacin/organic acid mix; about 250% w/w of the niacin/organic acid mix; about 275% w/w of the niacin/organic acid mix; about 300% w/w of the niacin/organic acid mix; about 325% w/w of the niacin/organic acid mix; about 350% w/w of the niacin/organic acid mix; about 375% w/w of the niacin/organic acid mix; about 400% w/w of the niacin/organic acid mix; about 425% w/w of the niacin/organic acid mix; about 450% w/w of the niacin/organic acid mix; about 475% w/w of the niacin/organic acid mix; about 500% w/w of the niacin/organic acid mix.

B. Reduced Variability of Niacin Absorption Between Fasted and Non-Fasted States

It has been reported that in the presence of food, absorption of niacin can be significantly affected and the therapeutic or flushing and other dysphoric effects can be attenuated when niacin is taken with food.

It is understood that niacin has extensive and saturable first pass metabolism. It is further understood that plasma level niacin concentrations in the general circulation are dose dependent and highly variable. Such variability may become more apparent in the presence of food, where absorption of niacin can be significantly reduced and the therapeutic or abuse deterrent effects of niacin can be attenuated when niacin is taken with food. Several factors may contribute to this effect of food presence on niacin absorption including the food's effect on niacin dispersibility and/or dissolution, and food's effect on gastrointestinal pH. For example, in the presence of food the rapid dispersibility and dissolution of niacin may be compromised, leading to slower absorption and more efficient first pass metabolism. In some embodiments, compositions and methods of the present invention achieve reduced variability of niacin absorption by enhancing niacin's dispersibility and/or dissolution, and/or by controlling the microenvironmental pH of gastrointestinal mucosa. In some embodiments, compositions and methods of the present invention provide a highly reproducible effect of niacin regardless of whether administered under fasted or fed condition.

C. Uses of Modified Niacin

Niacin prepared according to some embodiments of the present invention may be used in any suitable pharmaceutical composition. In some embodiments, niacin prepared according to the present invention is used in immediate and controlled release oral solid dosage forms such as conventional tablets and capsules, quick dissolve doses, sublingual tablets, buccal tablets, suppositories, pellets, effervescent preparations, soft chew and/or chewable tablets.

In some embodiments, niacin is included in a pharmaceutical composition as an aversive agent. In some embodiments, niacin is included in a pharmaceutical composition as a therapeutic agent. Niacin prepared according to some embodiments of the present invention may reduce variability in niacin absorption and therefore provide more consistent aversive and/or therapeutics effects of niacin. In certain embodiments, in accordance with the teachings herein, niacin can provide enhanced abuse deterrence, particularly when included in a dosage form of the present invention which includes a drug susceptible to abuse and the drug is abused by excess oral consumption while the abuser is in a fed state.

1. Systemic Aversive Agent

In some embodiments, niacin is used in an orally administered dose form as an aversive agent in combination with a drug susceptible to abuse. Drug abusers typically may take a prescription oral solid dosage form containing one or more drugs susceptible to abuse and crush, shear, grind, chew, dissolve, heat, extract or otherwise tamper with or damage the dosage unit so that a significant portion or even the entire amount of the active drug becomes available for administration by 1) injection, 2) nasal snorting, and/or 3) oral consumption in amounts exceeding the typical therapeutic dose for such drugs.

Niacin is well tolerated when combination opioids/niacin product candidates are administered at recommended doses. However, when an excess of tablets are swallowed above the recommended dose, the resulting higher niacin dose may induce temporary dysphoric effects, e.g., systemic effects, and a combination of unpleasant symptoms result, including flushing, itching, sweating and/or chills, headache and a general feeling of discomfort.

2. Local Aversive Agent

In some embodiments, the present invention can include one or more mucous membrane irritants, and/or respiratory passageway (e.g., oral or nasal) tissue irritants, and/or irritants to oral cavity or throat including the pharynx.

In one embodiment, suitable mucous membrane irritants and/or respiratory (e.g., oral or nasal) passageway tissue irritants include compounds that are generally considered pharmaceutically inactive, yet can induce irritation. Such compounds include, but are not limited to modified niacin, such as that described herein, and surfactants, including in certain embodiments anionic surfactants as described herein below.

In one embodiment, suitable surfactants include sodium lauryl sulfate, poloxamer, sorbitan monoesters and glyceryl monooleates. Other suitable compounds are believed to be within the knowledge of a practitioner skilled in the relevant art, and include certain vasodilators, and can be found in the Handbook of Pharmaceutical Excipients, 4th Ed. (2003), the entire content of which is hereby incorporated by reference.

As discussed above, some embodiments of the present invention can include niacin and sodium lauryl sulfate. Because modified niacin possesses nasal irritation characteristics, an additional result of such a composition is that a stabilized surfactant—(e.g., sodium lauryl sulfate) niacin nanoparticle will have more intense aversive effects by dispersing and dissolving significantly faster into the nasal mucosa than a simple admixture of these ingredients.

Upon contact with a mucous membrane, irritants may induce temporary discomfort, pain and/or irritation of the membranes and/or tissues to thereby deter abuse. For example, if inhaled by snorting, the mucous membrane in the nasal passageway will be irritated and result in significant discomfort and/or pain to the individual.

In another embodiment, the irritant can be pharmaceutically active. In such embodiments, the irritant can include one or more members of the vanilloid family and derivatives thereof, including capsaicin.

Examples of suitable irritants may be of natural or synthetic origin and include mustard, for example, allyl isothiocyaanate and p-hydroxybenzyl isothiocyanate; capsaicinoids such as capsaicin, dihydrocapsaicin, nordihydrocapsaiscin, homocapsaicin, and homodihydrocapsaicin, mint; aspirin; and acids such as acids with one or more carboxyl moieties such as formic acid, acetic acid, propionic acidy, butyric acid, valeric acid, caproic acid, caprillic acid, capric acid, oxalic acid, malonic acid, succicnic acid, glutaric acid, adipic acid, maleic acid, fumaric acid, and citric acid.

In one embodiment of the present invention, the irritant can be present in an amount of from 1 to 20 percent by weight on a solid basis, preferably 1 to 10 percent by weight on a solid basis. In another embodiment, the amount of irritant can be present in an amount of 5 to 15 percent by weight. In another embodiment, the irritant can be present in an amount of at least 5 percent by weight. In yet another embodiment, the irritant can be present in an amount from 1 to 5 percent by weight. In another embodiment, the amount of irritant can be present in an amount from 1 to 3 percent by weight.

In certain embodiments, the irritant can deter abuse of a dosage form when a potential abuser tampers with a dosage form of the present invention. Specifically, in such embodiments, when an abuser crushes the dosage form, the irritant is exposed. The irritant discourages inhalation (e.g., oral or nasal) of the crushed dosage form by inducing pain and/or irritation of the abuser's mucous membrane and/or respiratory passageway tissue. In one embodiment, the irritant discourages inhalation (e.g., via breathing through the mouth or via snorting through the nose) by inducing pain and/or irritation of the abuser's respiratory (e.g., nasal or oral) passageway tissue.

In one embodiment, the present invention includes one or more mucous membrane irritants to cause irritation of mucous membranes located anywhere on or in the body, including membranes of the mouth, eyes, nose and intestinal tract. Such compositions can deter abuse via oral, intra-ocular, rectal, or vaginal routes.

The above-described irritants can be further optimized as necessary or desired in terms of concentration, irritation severity, etc.

3. Therapeutic

Niacin prepared according to some embodiments of the present invention may be used as a therapeutic agent in pharmaceutical compositions. Niacin is commercially available in both immediate release form (Niacor®) and controlled release form (Niaspan®). The current invention may also be used in various release configurations such as but not limited to immediate release, controlled release, sustained release, delayed release, pulsatile release or a combination of release modes. Modified niacin may provide therapeutic effects as a vitamin in the treatment of pellagra, in cholesterol reduction and high density lipoprotein elevation, and is also reported to be used in the treatment of schizophrenia, pediatric behavior disorders, alcoholism, arthritis, Type 1 diabetes, and cancer.

D. Other Constituents

Any drug, therapeutically acceptable drug salt, drug derivative, drug analog, drug homologue, or polymorph can be used in the present invention. In one embodiment, the drug is an orally administered drug. In certain embodiments, drugs susceptible to abuse are used. Drugs commonly susceptible to abuse include psychoactive drugs and analgesics, including but not limited to opioids, opiates, stimulants, tranquilizers, narcotics and drugs that can cause psychological and/or physical dependence. In one embodiment, the drug for use in the present invention can include amphetamines, amphetamine-like compounds, amphetamine and methyl phenidate or combinations thereof. In another embodiment, the present invention can include any of the resolved isomers of the drugs described herein, and/or salts thereof.

A drug for use in the present invention which can be susceptible to abuse can be one or more of the following: alfentanil, amphetamines, buprenorphine, butorphanol, carfentanil, codeine, dezocine, dihydrocodeine, dihydromorphine, diphenoxylate, diprenorphine, etorphine, fentanyl, hydrocodone, hydromorphone, β-hydroxy-3-methylfentanyl, levo-α-acetylmethadol, levorphanol, lofentanil, meperidine, methadone, methylphenidate, morphine, nalbuphine, nalmefene, oxycodone, oxymorphone, pentazocine, pethidine, propoxyphene, remifentanil, sufentanil, tapentadol, tilidine and tramodol, salts, derivatives, analogs, homologues, polymorphs thereof, and mixtures of any of the foregoing.

In some embodiments, a drug for use with the present invention which can be susceptible to abuse includes one or more of the following: allobarbital, allylprodine, alprazolam, amphetamine, amphetaminil, amobarbital, anileridine, barbital, bezitramide, bromazepam, diazepine, brotizolam, butobarbital, camazepam, cathine/D-norpseudoephedrine, chlordiazepoxide, clobazam, clonazepam, clorazepate, clotiazepam, cloxazolam, cyclobarbital, cyclorphan, cyprenorphine, delorazepam, diampromide, diazepam, dihydromorphine, dimenoxadol, dimephetamol, dimethylthiambutene, dioxaphetyl butyrate, dipipanone, dronabinol, eptazocine, estazolam, ethylloflazepate, etonitrazene, fencamfamine, fenethylline, fenproporex, fludiazepam, flunitrazepam, flurazepam, halazepam, haloxazolam, hydroxypethidine, isomethadone, hydroxymethylmorphinan, ketazolam, ketobemidone, lormetazepam, mazindol, medazepam, meprobamate, meptazinol, metazocine, methaqualone, methylphenobarbital, methyprylon, metopon, midazolam, modafinil, myrophine, narceine, nimetazepam, nordazepam, norlevorphenol, oxazepam, oxazolam, plants and plant parts of the plants belonging to the species Papaver somniferum, papavereturn, pernoline, pentobarbital, phenadoxone, phenomorphan, phenazocine, phenoperidine, piminodine, pholcodeine, phenmetrazine, phentermine, pinazepam, piritramide, prazepam, profadol, proheptazine, promedol, properidine, secbutabarbital, secobarbital, temazepam, tapetadol tetrazepam, tramadol triazolam, vinylbital, each optionally in the form of corresponding stereoisomeric compounds and corresponding derivatives, including esters, ethers, salts and solvates. In one embodiment, a pharmaceutical composition of the present invention includes one or more opioids such as hydrocodone, morphine and oxycodone and/or salts thereof, as the therapeutically active ingredient. Typically when processed into a suitable dosage form, as described in more detail below, the drug can be present in such dosage forms in an amount normally prescribed, typically about 0.5 to about 25 percent on a dry weight basis, based on the total weight of the formulation.

With respect to analgesics in unit dose form, such an amount can be typically from about 5, 25, 50, 75, 100, 125, 150, 175 or 200 mg. More typically, the drug can be present in an amount from 5 to 500 mg or even 5 to 200 mg. In other embodiments, a dosage form contains an appropriate amount of drug to provide a therapeutic effect.

In another embodiment, the present invention includes one or more constituents or drugs which are not typically susceptible to abuse in addition to a drug which is susceptible to abuse, described above. In certain embodiments, the one or more additional drugs which are not typically susceptible to abuse can have an abuse deterrent effect (as described in more detail below) when administered in combination with a drug which is susceptible to abuse. In one embodiment of a dosage form of the present invention which includes a drug that is susceptible to abuse, the one or more additional drugs which can induce an abuse deterrent effect can be included in the dosage form in a sub-therapeutic or sub-clinical amount.

As used herein, “sub-therapeutic” or “sub-clinical” refer to an amount of a referenced substance that if consumed or otherwise administered, is insufficient to induce an abuse deterrent effect (e.g., nausea) in an average subject or is insufficient to meet or exceed the threshold dose necessary for inducing an abuse deterrent effect.

Accordingly, when an embodiment of a dosage form of the present invention is administered in accordance with a health care provider prescribed dosage and/or manner, the one or more additional drugs which can induce an abuse deterrent effect will not be administered in an amount sufficient to induce an abuse deterrent effect. However, when a certain embodiment of the present invention is administered in a dose and/or manner that is different from a health care provider prescribed dose, (i.e., the drug is abused or the dosage form is tampered with) the content of a formulation which can cause an abuse deterrent effect according to the present invention will be sufficient to induce an abuse deterrent effect. Suitable examples of other constituents which can be administered in sub-therapeutic amounts in the present invention include unmodified niacin, atropine sulfate, naltrexone hydrochloride, homatropine methylbromide, sildenafil citrate, nifedipine, zinc sulfate, dioctyl sodium sulfosuccinate and capsaicin.

Pharmaceutical compositions of the present invention may also include other ingredients, as explained in detail in U.S. Publication No. 2006-0177380, the entire contents of which are incorporated by reference herein, and where it is understood that the modified niacin of the present invention may be substituted for or used in combination with the niacin described therein. Such components include but are not limited to fillers/diluents, disintegrants, glidants, lubricants, excipients, opioid antagonists, and/or sequestration polymers.

E. Dosage Forms

In certain embodiments, a dosage form includes niacin in accordance with the teachings herein and/or in accordance with U.S. Publication No. 2006-0177380, the entire content of which is incorporated by reference.

Suitable formulations and dosage forms of the present invention include but are not limited to powders, caplets, pills, suppositories, gels, soft gelatin capsules, capsules and compressed tablets manufactured from a pharmaceutical composition of the present invention. The dosage forms can be any shape, including regular or irregular shape depending upon the needs of the artisan.

Compressed tablets including the pharmaceutical compositions of the present invention can be direct compression tablets or non-direct compression tablets. In one embodiment, a dosage form of the present invention can be made by wet granulation, and dry granulation (e.g., slugging or roller compaction). The method of preparation and type of excipients are selected to give the tablet formulation desired physical characteristics that allow for the rapid compression of the tablets. After compression, the tablets must have a number of additional attributes such as appearance, hardness, disintegrating ability, and an acceptable dissolution profile.

Choice of fillers and other excipients typically depend on the chemical and physical properties of the drug, behavior of the mixture during processing, and the properties of the final tablets. Adjustment of such parameters is understood to be within the general understanding of one skilled in the relevant art.

The manufacture of a dosage form of the present invention can involve direct compression and wet and dry granulation methods, including slugging and roller compaction.

Accordingly, and as described further below, a directly compressible pharmaceutical composition of the present invention can be designed following the teachings set forth herein that can deter one or more of a) parenteral abuse of a drug, b) inhalation abuse of a drug, and c) oral abuse of a drug.

Such compositions and dosage forms are formed according to the present invention are described. Steps for making the compositions or dosage forms include the step of providing one or more drugs and/or analgesics described above and providing niacin as described above.

As used throughout, the term “about” is understood to mean+10% of the value referenced. For example, “about 100” is understood to literally mean 90 to 110.

### EXAMPLES

### Example 1

Samples of reduced particle niacin and amorphous niacin were compared to a standard niacin grade by two dissolution techniques. The samples included the following: Niacin Special (60 μm); Micronized niacin (4 μm); Nanoparticulate niacin (250 nm); and Amorphous niacin #1 (limited only by the size of a single niacin molecule).

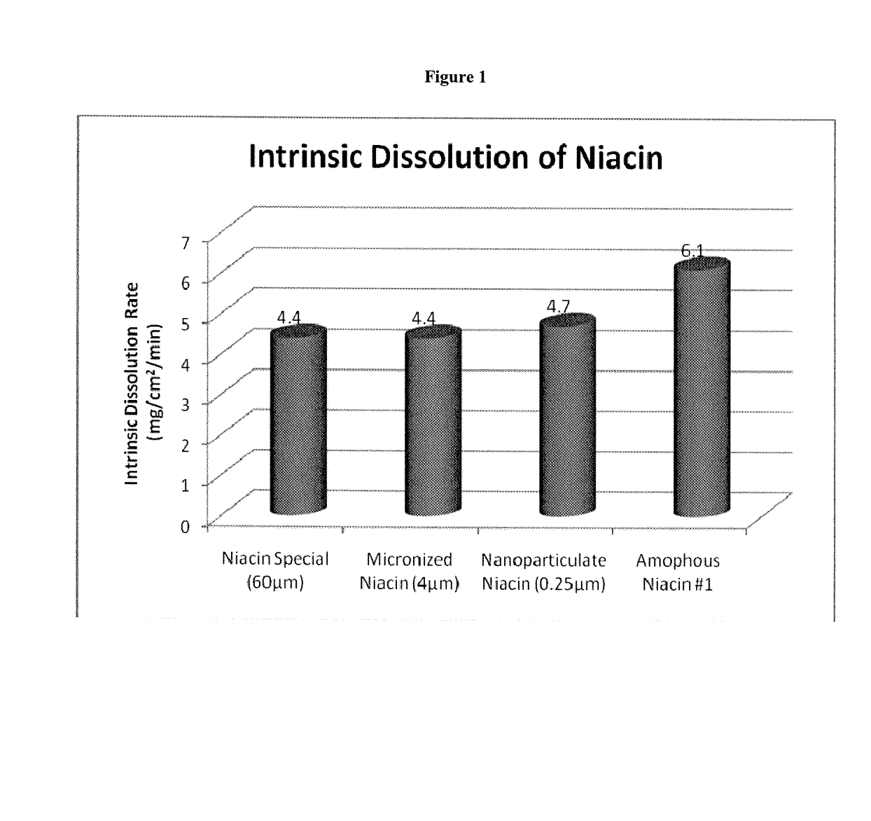

FIG. 1 shows the intrinsic dissolution of niacin under sink conditions for micronized niacin, nanoparticulate niacin and an amorphous niacin sample. Sample powders were placed in a die and compressed such that a fixed surface area of compact was exposed at the surface of the die. The compacted dies were placed in 500 mL of 0.1N hydrochloric acid solution at 37° C. with a paddles speed of 50 rpm in a standard dissolution apparatus. Samples were collected every minute for five minutes and intrinsic dissolution rates were calculated. Corrections were made to samples where adjuvants have been added to niacin (nanoparticle niacin contains 90% niacin; amorphous niacin contains 30% niacin).

Because the intrinsic dissolution test normalizes the exposed surface area, it was anticipated that micronized niacin would have a similar dissolution rate compared to the reference niacin sample, Niacin special. However, the intrinsic dissolution rate for nanoparticle showed a slightly faster intrinsic dissolution (7% faster) which may be a conversion of some crystalline to amorphous regions in the niacin nanoparticle. As shown in FIG. 1, the amorphous niacin sample demonstrates that the amorphous form of niacin has a faster dissolution rate (39% faster) when compared to the standard crystalline form.

### Example 2

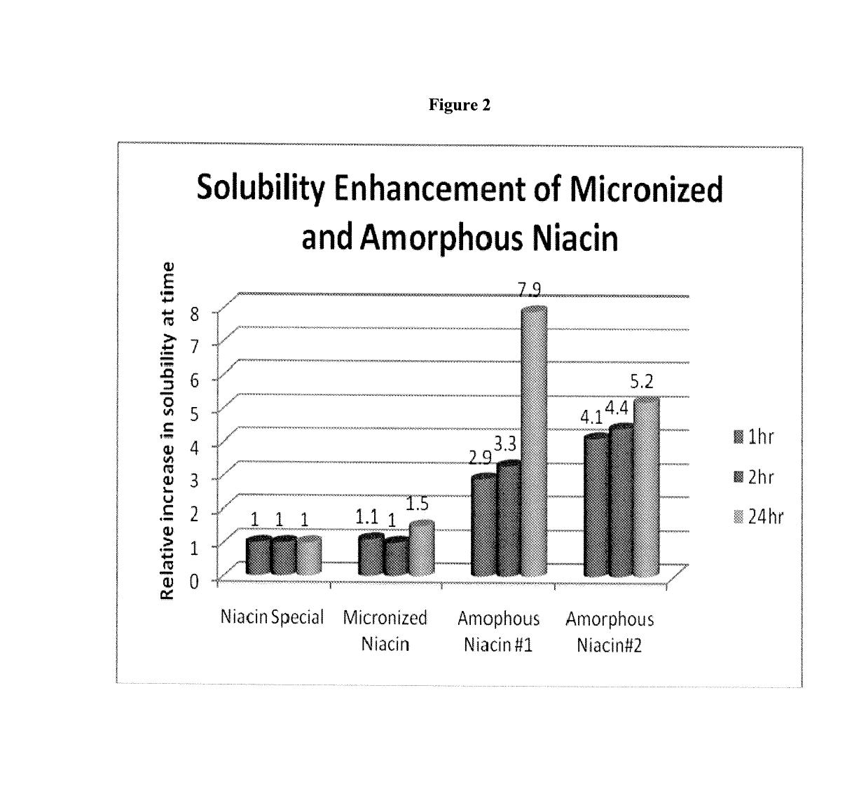

FIG. 2 demonstrates the kinetic solubility under non-sink conditions (concentration rate-limited) for two amorphous niacin samples and a micronized niacin sample compared to a niacin reference. Amorphous niacin #1 contains 30% niacin and 70% amino methacrylate copolymer. Amorphous niacin #2 contains 30% niacin and 70% poloxamer. The samples were compared at 1, 2 and 24 hrs and values for the solubility enhanced samples are shown relative to the reference at each given time (value=AUC sample @time (t)/AUC reference @time (t)).

At the early time points, micronized niacin kinetic solubility is equal to the reference but does show a 50% increase after 24 hours. However, both amorphous samples (30% Niacin in two different polymers) demonstrate significantly higher kinetic solubility at all time points compared to the reference niacin. Amorphous niacin #1 shows the greatest increase of kinetic solubility at 790% relative to the reference.

### Example 3

In a Phase I single-center, randomized, double-blind study, micronized niacin (median particle size approximately 4 μm) was compared to standard niacin (median particle size approximately 60 μm) in healthy male and female adult subjects under fed conditions.

A total of 49 subjects were enrolled. Each subject received 480 mg of micronzed niacin fasted and 480 mg of regular niacin taken orally in tablet form separated by a 24-hour washout period between each dose. As identified in the randomization schedule for each subject, subjects took each dose of study drug immediately following a standardized high-fat breakfast.

During the study, each subject completed a 100 point Visual Analog Scale (VAS) “Like/Dislike” Rating Scale at dosing and for up to 6 hours post-dosing where subjects were asked “Do you like or dislike the drug effect you are feeling now?”

As a subset in this study, subjects were identified as “high responders” who recorded at least one VAS score less than or equal to −20, where high negative scores are indicative of high subject disliking from niacin-induced adverse events such as flushing and pruritis. Of the six high responder subjects, five patients demonstrated lower VAS scores for micronized niacin compared to standard niacin. The mean peak disliking effect (Emin) for high responders taking 480 mg micronized niacin is −28.7 mm and the mean time to peak disliking is 2.25 hrs, whereas the mean peak disliking effect for high responders taking 480 mg standard niacin is −15.3 mm and the mean time to peak disliking is 3.0 hrs.

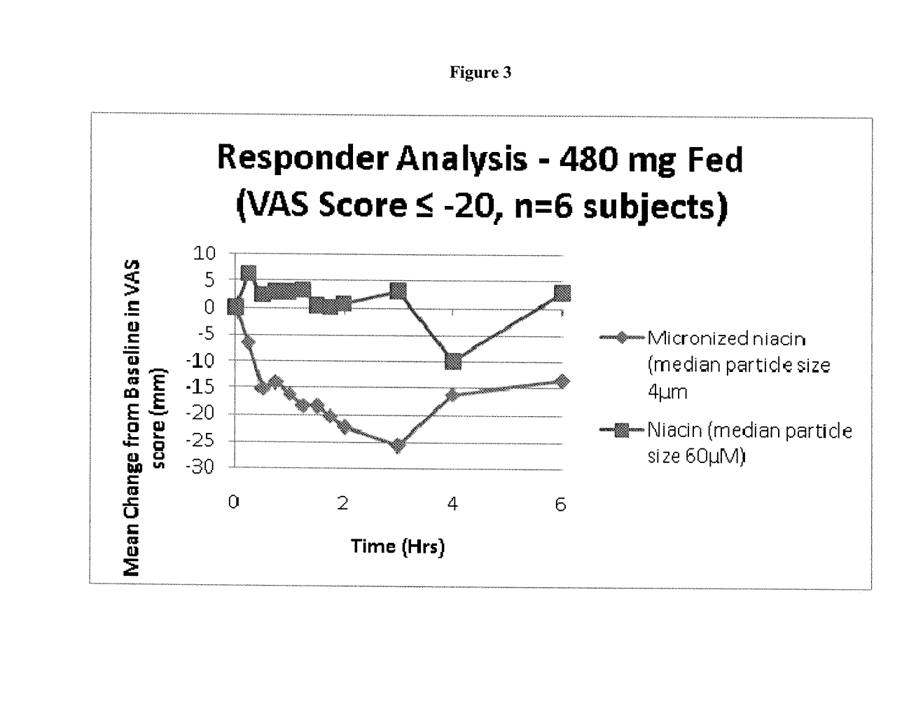

FIG. 3 shows the mean change from baseline in VAS scores over 6 hours. The data show that micronized niacin has a more substantial disliking effect (89%) with faster time to peak disliking (25%) than standard particle size niacin in the presence of food. These data suggest that reduction of niacin particle size in the presence of food results in improving the effectiveness of niacin as an oral aversive agent in drug abuse deterrence and that further particle size reduction such as nanoparticle (approximately 10-1000 nm) or to an amorphous molecular dispersion may provide more significant bioavailability of niacin in the fed state.

### Example 4

A pharmaceutical composition of the following formulation was prepared:

| Component | Weight (mg)/tablet |
|---|---|
| Oxycodone hydrochloride | 5 |
| Polyethylene oxide | 25 |
| Microcrystalline cellulose | 320 |
| Niacin, micronized | 30 |
| Sodium lauryl sulfate | 7 |
| Crospovidone | 100 |
| Colloidal silicon dioxide | 2 |
| Magnesium stearate | 1 |
| Total | 490 |

The niacin particles of the formulation have a diameter of 4 μm.

### Example 5

A pharmaceutical composition of the following formulation was prepared:

| Component | Weight (mg)/tablet |
|---|---|
| Hydrocodone bitartrate | 5 |
| Polyethylene oxide | 30 |
| Microcrystalline cellulose | 270 |
| Niacin, nanoparticle | 30 |
| Sodium lauryl sulfate | 25 |
| Crospovidone | 100 |
| Citric acid | 150 |
| Colloidal silicon dioxide | 2 |
| Magnesium stearate | 3 |
| Total | 615 |

The niacin particles of the formulation have a diameter of 250 nm.

### Example 6

A pharmaceutical composition of the following formulation was prepared:

| Component | Weight (mg)/tablet |
|---|---|
| Hydromorphone hydrochloride | 4 |
| Polyethylene oxide | 20 |
| Microcrystalline cellulose | 200 |
| Niacin:amino methacrylate | 100 |
| copolymer dispersion 30:70 | |
| Sucrose stearate | 18 |
| Tartaric acid | 75 |
| Crospovidone | 80 |
| Colloidal silicon dioxide | 2 |
| Magnesium stearate | 2 |
| Total | 500 |

### Example 7

A pharmaceutical composition of the following formulation was prepared:

| Component | Weight (mg)/tablet |
|---|---|
| Oxymorphone hydrochloride | 10 |
| Polyethylene oxide | 25 |
| Microcrystalline cellulose | 300 |
| Niacin:Citric Acid cocrystal (1:2) | 75 |
| Sodium lauryl sulfate | 15 |
| Crospovidone | 100 |
| Cab-O-Sil | 2 |
| Magnesium stearate | 3 |
| Total | 515 |

In light of the teachings set forth herein, an embodiment of the invention having the above described composition can be made.

It will be appreciated by persons skilled in the art that numerous variations and/or modifications may be made to the invention shown in the specific embodiments without departing form the spirit and scope of the invention as broadly described. Further, each and every reference cited above is hereby incorporated by reference as if fully set forth herein.

We claim:

1. A pharmaceutical composition comprising a stable nanoparticle comprising niacin, wherein the nanoparticle has a particle size of about 100 nm to about 300 nm.

2. The pharmaceutical composition of claim 1, wherein nanoparticle further comprises surface stabilizing ingredients.

3. The pharmaceutical composition of claim 2, wherein the surface stabilizing ingredients comprise one or more of a surfactant, a powder flow aid, or a polymer.

4. The pharmaceutical composition of claim 2, wherein the surface stabilizing ingredient comprises sodium lauryl sulfate.

5. The pharmaceutical composition of claim 1, wherein the niacin is amorphous.

6. The pharmaceutical composition of claim 1, further comprising an organic acid.

7. The pharmaceutical composition of claim 6, wherein the niacin and organic acid are mixed at a particulate level.

8. The pharmaceutical composition of claim 6, wherein the niacin and organic acid form cocrystals.

9. The pharmaceutical composition of claim 6, wherein at least a portion of the niacin and organic acid forms a molecular dispersion.

10. The pharmaceutical composition of claim 6, wherein the organic acid has a pKa of about 2.0 to about 6.5.

11. The pharmaceutical composition of claim 6, wherein the organic acid comprises at least one of oxalic acid, glycine, taurine, citric acid, pyruvic acid, alanine, maleic acid, glutaric acid, or tartaric acid.

12. The pharmaceutical composition of claim 6, wherein the ratio of niacin to organic acid is from about 1:5 to about 5:1.

13. The pharmaceutical composition of claim 6, wherein the ratio of niacin to organic acid is from about 1:3 to about 3:1.

14. A pharmaceutical composition comprising amorphous niacin, wherein the amorphous niacin comprises a solid dispersion of niacin and a polymer.

15. The pharmaceutical composition of claim 14, wherein the polymer comprises amino methacrylate copolymer.

16. The pharmaceutical composition of claim 14, wherein the polymer comprises poloxamer.

17. The pharmaceutical composition of claim 14, wherein the polymer is present in an amount of up to about 1000% w/w of niacin.

18. The pharmaceutical composition of claim 14, wherein the polymer is present in an amount of about 20% w/w to about 400% w/w of niacin.

19. The pharmaceutical composition of claim 14, wherein the niacin comprises a stable nanoparticle.

20. The pharmaceutical composition of claim 14, further comprising an organic acid.

21. The pharmaceutical composition of claim 20, wherein the niacin and organic acid are mixed at a particulate level.

22. The pharmaceutical composition of claim 20, wherein the niacin and organic acid form cocrystals.

23. The pharmaceutical composition of claim 20, wherein at least a portion of the niacin and organic acid forms a molecular dispersion.

24. The pharmaceutical composition of claim 20, wherein the organic acid has a pKa of about 2.0 to about 6.5.

25. The pharmaceutical composition of claim 20, wherein the organic acid comprises at least one of oxalic acid, glycine, taurine, citric acid, pyruvic acid, alanine, maleic acid, glutaric acid, or tartaric acid.

26. The pharmaceutical composition of claim 20, wherein the ratio of niacin to organic acid is from about 1:5 to about 5:1.

27. The pharmaceutical composition of claim 20, wherein the ratio of niacin to organic acid is from about 1:3 to about 3:1.

28. The pharmaceutical composition of claim 14, wherein the polymer comprises a low molecular weight polymer.

29. The pharmaceutical composition of claim 14, wherein the polymer comprises a water soluble polymer.

30. The pharmaceutical composition of claim 14, wherein the polymer comprises a polymer soluble in low pH media.

31. A pharmaceutical composition comprising,
(a) an opioid analgesic;
(b) niacin in an amount sufficient to cause flushing if greater than a prescribed amount of the analgesic of the pharmaceutical composition is ingested;
wherein the niacin comprises a stable nanoparticle.

32. The composition of claim 31, wherein the nanoparticle further comprises surface stabilizing agents.

33. The composition of claim 31, wherein the niacin is amorphous.

34. The composition of claim 31, further comprising an organic acid.

35. The composition of claim 34, wherein the niacin and organic acid are cocrystals.

36. The composition of claim 31, wherein the composition is in unit dose form.

37. The composition of claim 31, wherein the composition is in a caplet, capsule, pill, gel, soft gelatin capsule, or compressed tablet form.

38. The composition of claim 31, wherein the analgesic is present in an amount of about 5 mg to about 200 mg on a solid weight basis.

39. The composition of claim 31, wherein the analgesic comprises hydrocodone or a therapeutically acceptable salt thereof.

40. The composition of claim 31, wherein the analgesic comprises oxycodone or a therapeutically acceptable salt thereof.

41. The composition of claim 31, further comprising a gel forming polymer

42. The composition of claim 31, further comprising a nasal tissue irritant.

43. A pharmaceutical composition comprising
(a) a drug susceptible to abuse; and
(b) niacin in an amount sufficient to cause flushing if greater than a prescribed amount of the drug of the pharmaceutical composition is ingested;
wherein the niacin comprises a stable nanoparticle.

44. The composition of claim 43, wherein the nanoparticle further comprises surface stabilizing agents.

45. The composition of claim 43, wherein the niacin is amorphous.

46. The composition of claim 43, further comprising an organic acid.

47. The composition of claim 46, wherein the niacin and organic acid are cocrystals.

48. The composition of claim 43, wherein the composition is in unit dose form.

49. The composition of claim 43, wherein the composition is in a caplet, capsule, pill, gel, soft gelatin capsule, or compressed tablet form.

50. The composition of claim 43, further comprising a gel forming polymer

51. The composition of claim 43, further comprising a nasal tissue irritant.

52. A pharmaceutical composition comprising
(a) a drug susceptible to abuse; and
(b) niacin in a sub-therapeutic amount;
wherein the niacin comprises a stable nanoparticle.

53. The composition of claim 52, wherein the nanoparticle further comprises surface stabilizing agents.

54. The composition of claim 52, wherein the niacin is amorphous.

55. The composition of claim 52, further comprising an organic acid.

56. The composition of claim 55, wherein the niacin and organic acid are cocrystals.

57. The composition of claim 52, wherein the composition is in unit dose form.

58. The composition of claim 52, wherein the composition is in a caplet, capsule, pill, gel, soft gelatin capsule, or compressed tablet form.

59. The composition of claim 52, further comprising a gel forming polymer

60. The composition of claim 52, further comprising a nasal tissue irritant.

61. A pharmaceutical composition comprising
(a) a drug susceptible to abuse; and
(b) amorphous niacin in an amount sufficient to cause flushing if greater than a prescribed amount of the drug of the pharmaceutical composition is ingested;
wherein the amorphous niacin comprises a solid dispersion of niacin and a polymer.

62. The pharmaceutical composition of claim 61, wherein the polymer comprises amino methacrylate copolymer.

63. The pharmaceutical composition of claim 61, wherein the polymer comprises poloxamer.

64. The pharmaceutical composition of claim 61, wherein the polymer is present in an amount of up to about 1000% w/w of niacin.

65. The pharmaceutical composition of claim 61, wherein the polymer is present in an amount of about 20% w/w to about 400% w/w of niacin.

66. The pharmaceutical composition of claim 61, wherein the niacin comprises a stable nanoparticle.

67. The pharmaceutical composition of claim 61, further comprising an organic acid.

68. The pharmaceutical composition of claim 67, wherein the niacin and organic acid are mixed at a particulate level.

69. The pharmaceutical composition of claim 67, wherein the niacin and organic acid form cocrystals.

70. The pharmaceutical composition of claim 61, wherein the composition is in unit dose form.

71. The pharmaceutical composition of claim 61, wherein the composition is in a caplet, capsule, pill, gel, soft gelatin capsule, or compressed tablet form.

72. The pharmaceutical composition of claim 61, further comprising a gel forming polymer

73. The pharmaceutical composition of claim 61, further comprising a nasal tissue irritant.

74. A pharmaceutical composition comprising
(a) a drug susceptible to abuse;
(b) niacin in an amount sufficient to cause flushing if greater than a prescribed amount of the drug of the pharmaceutical composition is ingested; and
(c) an organic acid.

75. The pharmaceutical composition of claim 74, wherein the niacin and organic acid are coprocessed.

76. The pharmaceutical composition of claim 74, wherein the niacin and organic acid form cocrystals.

77. The pharmaceutical composition of claim 74, wherein at least a portion of the niacin and organic acid forms a molecular dispersion.

78. The pharmaceutical composition of claim 74, wherein the organic acid has a pKa of about 2.0 to about 6.5

79. The pharmaceutical composition of claim 74, wherein the organic acid comprises at least one of oxalic acid, glycine, taurine, citric acid, pyruvic acid, alanine, maleic acid, glutaric acid, or tartaric acid.

80. The pharmaceutical composition of claim 74, wherein the ratio of niacin to organic acid is from about 1:5 to about 5:1.

81. The pharmaceutical composition of claim 74, wherein the ratio of niacin to organic acid is from about 1:3 to about 3:1.

82. The pharmaceutical composition of claim 74, wherein the niacin is amorphous.

83. The pharmaceutical composition of claim 82, wherein the amorphous niacin comprises a solid dispersion of niacin and a polymer.

84. The pharmaceutical composition of claim 74, wherein the niacin comprises a stable nanoparticle.

85. The pharmaceutical composition of claim 84, wherein the nanoparticle further comprises surface quenching agents.

86. The pharmaceutical composition of claim 74, wherein the composition is in unit dose form.

87. The pharmaceutical composition of claim 74, wherein the composition is in a caplet, capsule, pill, gel, soft gelatin capsule, or compressed tablet form.

88. The pharmaceutical composition of claim 74, further comprising a gel forming polymer.

89. The pharmaceutical composition of claim 74, further comprising a nasal tissue irritant.
